SEQUENCE LISTING (5 sequences)

SEQ ID NO: 1 - RNA, 24 nt, Homo sapiens
  misc_feature: hsa-miR-1291
uggcccugacugaagaccagcagu

SEQ ID NO: 2 - RNA, 22 nt, Homo sapiens
  misc_feature: hsa-miR-1-3p
uggaauguaaagaaguauguau

SEQ ID NO: 3 - RNA, 22 nt, Homo sapiens
  misc_feature: hsa-miR-214-3p
acagcaggcacagacaggcagu

SEQ ID NO: 4 - RNA, 22 nt, Homo sapiens
  misc_feature: hsa-miR-375
uuuguucguucggcucgcguga

SEQ ID NO: 5 - RNA, 22 nt, Homo sapiens
  misc_feature: hsa-let-7a-5p
ugagguaguagguuguauaguu

(19) United States
(12) Patent Application Publication
(10) Pub. No.: US 2023/0287504 A1
(43) Pub. Date: Sep. 14, 2023

(54) A PERIPHERAL BLOOD MIRNA MARKER FOR DIAGNOSIS OF NON-SMALL CELL LUNG CANCER

(71) Applicants: Mirxes (Hangzhou) Biotechnology Co., Ltd., Zhejiang (CN); Zhejiang Cancer Hospital, Zhejiang (CN)

(72) Inventors: Ruiyang Zou, Zhejiang (CN); Dan Su, Zhejiang (CN); He Cheng, Zhejiang (CN); Lisha Ying, Zhejiang (CN); Lihan Zhou, Zhejiang (CN)

(21) Appl. No.: 17/293,382

(22) PCT Filed: Nov. 11, 2019
(86) PCT No.: PCT/CN2019/117176

§ 371 Date: May 12, 2021

(30) Foreign Application Priority Data
Nov. 12, 2018 (CN) 201811336972.2

(51) Int. Cl. C12Q 1/6886

(52) U.S. Cl. CPC C12Q 1/6886; C12Q 2600/158; C12Q 2600/178; C12Q 2600/118

(57) ABSTRACT

Provided are peripheral blood miRNA markers for diagnosis of non-small cell lung cancer, wherein the peripheral blood miRNA markers comprise hsa-miR-1291, hsa-miR-1-3p, hsa-miR-214-3p, hsa-miR-375 or hsa-let-7a-5p. Five specific diagnostic markers suitable for diagnosing non-small cell lung cancer in Asian and Caucasian populations are validated on a large number of samples, and have higher population specificity compared to other miRNA markers previously reported. These five miRNA diagnostic markers are proposed for the first time and are shown to be more reliable than other miRNA molecular markers.

### FIELD OF THE INVENTION

The invention relates to the technical field of early detection of diseases, and in particular to a peripheral blood miRNA marker for diagnosis of non-small cell lung cancer.

### BACKGROUND OF THE INVENTION

Lung cancer is a leading cause of cancer deaths worldwide. According to statistics released by National Cancer Centre in 2016, 733,000 cases among 4.29 million newly developed cancer patients are due to lung cancer. Amongst the 2.8 million cancer deaths, lung cancer is responsible for 610,000 cases, making it the “first cancer” in China in name and in fact. Among them, non-small cell lung cancer (NSCLC) accounts for about 80% of all lung cancers, of which about 75% of patients are already in the middle-advanced stage when diagnosed, with a very low 5-year survival rate. Since the early symptoms of lung cancer are not obvious, 75% of lung cancer patients have suffered from local infiltration and distant metastasis at the time of visit, losing their chance for surgery. The current treatment has little effect on improving the overall survival rate of lung cancer, where the 5-year survival rate is approximately between 40% and 5% for lung cancer patients in stages II-IV and can be up to 92% for patients in stage I. Therefore, strengthening the screening for the high-risk population and improving the early diagnosis and treatment rate is the most effective way to reduce lung cancer mortality.

Chest X-rays and sputum smears are the most common techniques for lung cancer screening, and however their sensitivity is too low. Fiberoptic bronchoscopy or biopsy can directly examine the lesion and determine the nature of the pathology, but is invasive, making them difficult to be applied to a large-sample population. Low-dose spiral CT is currently considered to be the most effective technique for lung cancer screening, which is non-invasive and highly sensitive, and however has a false-positive rate of up to 96.4%, and the cost for screening is relatively high. There is therefore a need to develop a novel technique for early screening which is minimally invasive, economical, and highly sensitive and specific.

MicroRNAs (miRNAs) are a class of non-coding small RNAs of 19-25 nucleotides in length that have been discovered in recent years. They degrade a target gene mRNA or inhibits translation thereof mainly by completely or incompletely pairing with 3′UTR of the target gene, thereby involving in the regulation of life activities such as ontogenesis, cell apoptosis, proliferation and differentiation, and playing a similar role to oncogenes or tumor suppressor genes during tumor's development and progression. The expression profile of miRNAs has obvious tissue specificity, having a specific expression pattern in different tumors. These characteristics make miRNA possible to become a novel biological marker and therapeutic target for tumor diagnosis. Like known circulating nucleic acids (DNA and RNA), miRNAs are widely present in the serum of healthy persons at a high risk of lung cancer and tumor patients, and the type and number thereof will be changed with physiological condition and disease progression. Circulating miRNAs may be derived from apoptotic or necrotic cells, or from active release by cells and lysis of circulating cells. Most of these endogenous circulating miRNA molecules do not exist in free form, but form complexes with proteins and the like. Therefore, endogenous circulating RNA molecules have excellent resistance against RNase degradation and high stability. This property makes it possible for the use of circulating miRNAs as biomarkers for detection.

Many studies have reported abnormal expression of miRNAs in lung cancer. Although the existing studies have discovered many very promising serum miRNAs for early diagnosis of lung cancer, there is no uniform conclusion on miRNA markers for non-small cell lung cancer, since the tested samples comprise tissue, serum, plasma, etc., the detection method includes sequencing, amplification, hybridization, etc., the selection for the enrolled samples in the study is not strict; and there is lack of consistency in various factors. These results are inconsistent and cannot be mutually verified. Finally, serum miRNA biomarkers and a combination thereof that can be used for lung cancer screening have not been concluded.

The most critical reasons for above are as follows:
1. Deviation occurs during the selection, collection and preservation of patient and control samples. A different type of samples will inevitably bring uncertainty to the development and verification of biomarkers. The miRNAs in the peripheral blood are mainly secreted by the lung cancer-related cells to the extracellular environment, and the components thereof are inevitably different from that in the cells or in whole blood samples, and are also affected by other factors, such as the presence or absence of prior treatments. Most miRNAs are stably present in the peripheral blood of healthy and cancer subjects, and are secreted by various tissue cells in the body, the expression level of which would be affected by various non-cancerous factors such as environmental and genetic factors. In order to eliminate the impact, a large number of human samples need to be selected for research and development as well as verification to verify the authenticity of the biomarkers. At the same time, studies have shown that the peripheral blood samples would have a different biomarker content when isolated and stored by using different methods. The biomarkers, which are discovered by cancer tissue cells, by comparing to advanced cancers, by comparing among samples isolated and stored using different methods, as well as not developed and validated by a large number of samples, may all be false-positive results and may not necessarily withstand the verification by a large-scale experiment.
2. Due to a very small molecular weight, miRNAs have certain difficulties in detection. It has always been a problem that how to detect miRNAs stably and sensitively especially in peripheral blood where miRNAs have a low content. The limitation of some current high-throughput chips, sequencing and high-throughput RT-PCR screening methods includes poor stability, poor reproducibility, and low sensitivity, etc. In combination with using a small number of samples, it is easy to produce false-negative results during the research and development (R&D) phase, ignoring important miRNA biomarkers. At the same time, the instability of the technology also increases the uncertainty of the verification of biomarkers in independent samples, and it is easy to increase the probability of false-positives and false-negatives.

### SUMMARY OF THE INVENTION

The object of the present invention is to provide a peripheral blood miRNA marker for diagnosis of non-small cell lung cancer, where based on verification by a large number of samples, five specific diagnostic markers are explicitly identified to be suitable for non-small cell lung cancer in Asian and Caucasian population, showing a higher population specificity in relative to other miRNA markers reported internationally. All of these five miRNA diagnostic markers are first proposed and are more reliable than other miRNA molecular markers.

The technical solutions adopted by the present invention to solve the technical problem thereof are:

a peripheral blood miRNA marker for diagnosis of non-small cell lung cancer, comprising at least one of hsa-miR-1291, hsa-miR-1-3p, and hsa-miR-214-3p.

The peripheral blood miRNA marker further comprises one or both of hsa-miR-375 and hsa-let-7a-5p.

A peripheral blood miRNA marker for the diagnosis of non-small cell lung cancer wherein the peripheral blood miRNA marker comprises at least one miRNA selected from hsa-miR-1291, hsa-miR-1-3p, hsa-miR-214-3p, hsa-miR-375 and hsa-let-7a-5p.

In one embodiment, the peripheral blood miRNA marker is a combination of two of hsa-miR-1291, hsa-miR-1-3p, hsa-miR-214-3p, hsa-miR-375, and hsa-let-7a-5p. In one embodiment, the peripheral blood miRNA marker is a combination of three of hsa-miR-1291, hsa-miR-1-3p, hsa-miR-214-3p, hsa-miR-375, and hsa-let-7a-5p. In another embodiment, the peripheral blood miRNA marker is a combination of four of hsa-miR-1291, hsa-miR-1-3p, hsa-miR-214-3p, hsa-miR-375, and hsa-let-7a-5p. In another embodiment, the peripheral blood miRNA marker is a combination of five of hsa-miR-1291, hsa-miR-1-3p, hsa-miR-214-3p, hsa-miR-375, and hsa-let-7a-5p.

The peripheral blood is serum or plasma.

The expression of the peripheral blood miRNA marker is differentially regulated in the peripheral blood of a patient diagnosed with non-small cell lung cancer compared to that in a control sample. hsa-miR-1291, hsa-miR-1-3p, hsa-miR-214-3p are up-regulated in cancer patients, while hsa-miR-375 and hsa-let-7a-5p are both down-regulated in cancer patients.

The control sample is a subject not suffering from non-small cell lung cancer.

The non-small cell lung cancer comprises squamous cell lung cancer, and adenocarcinoma lung cancer.

A kit for diagnosis of non-small cell lung cancer, comprising at least one reagent for detecting the peripheral blood miRNA marker. The kit is for detecting the expression level of at least one miRNA selected from the group comprising hsa-miR-1291, hsa-miR-1-3p, hsa-miR-214-3p, hsa-miR-375, and hsa-let-7a-5p.

Use of the peripheral blood miRNA marker in the preparation of a diagnostic agent for non-small cell lung cancer for predicting the possibility for a subject to develop or have non-small cell lung cancer by a method, the method comprising:
detecting the presence of miRNAs in a peripheral blood sample obtained from the subject;
measuring the expression level of at least one miRNA selected from of hsa-miR-1291, hsa-miR-1-3p, hsa-miR-214-3p, hsa-miR-375, and hsa-let-7a-5p in the peripheral blood sample; and
using a score based on the previously measured miRNA expression level to predict the possibility that the subject will develop or have non-small cell lung cancer.

The score of the expression level of miRNA is calculated using a classification algorithm selected from the group consisting of: support vector machine algorithm, logistic regression algorithm, multinomial logistic regression algorithm, Fisher's linear discriminant algorithm, quadratic classifier algorithm, perceptron algorithm, k-nearest neighbor algorithm, artificial neural network algorithm, random forest algorithm, decision tree algorithm, naive Bayes algorithm, adaptive B ayes network algorithm, and an integrated learning method that combines multiple learning algorithms.

The classification algorithm is pre-trained using the expression level of a control.

Wherein the control is at least one selected from the group consisting of a control without non-small cell lung cancer and a non-small cell lung cancer patient.

Wherein the classification algorithm compares the expression level in the subject with that in the control and returns a mathematical score that identifies the possibility that the subject belongs to any one of the control groups.

Wherein the expression level of the miRNA is in any one of concentration, log(concentration), Ct/Cq, and Ct/Cq power of 2.

The non-small cell lung cancer comprises non-small cell lung cancer in various stages.

The subject comprises, but is not limited to, Asians and Caucasians.

There is no uniform conclusion on serum/plasma miRNA biomarkers for lung cancer in the current reports. These results are inconsistent, some of which are up-regulated, and some are down-regulated, and cannot be mutually verified. Finally, the serum miRNA biomarkers and a combination thereof that can be used for lung cancer screening have not yet been concluded. Examples of existing reports are as follows:

| Publication | Upregulated | Downregulated | Sample | Comments |
|---|---|---|---|---|
| Chen et al, Identification of ten | miR-20a, miR-24, | | Serum, 400 | Starting |
| serum microRNAs from a | miR-25, miR-145, | | NSCLC, 220 C | with 91 |
| genome-wide serum | miR-152, | | | miRNAs |
| microRNA expression profile | miR-199a-5p, | | | (qPCR) |
| as novel noninvasive | miR-221, miR-222, | | | |
| biomarkers for non-small cell | miR-223, miR-320 | | | |
| lung cancer diagnosis. | | | | |
| Wanget al, Identification and | miR-21 | miR-7a | Serum, 172 | Starting |
| Evaluation of Two Circulating | | | NSCLC, 133C | with 10 |
| microRNAs for Non-small | | | | miRNA |
| Cell Lung Cancer Diagnosis | | | | (qPCR) |
| Zaporozhchenkoet al, Profiling | miR-19a-3p, | miR-486-5p, | Serum, 50 | Starting |
| of 179 miRNA Expression | miR-19b-3p, | miR-144-5p, | NSCLC, 30 C | with 191 |
| in Blood Plasma of Lung | miR-130b-3p, | miR-20a-5p, | | miRNA |
| Cancer Patients | miR-30e-5p, | miR-150-5p, | | (qPCR) |
| and Cancer-Free Individuals. | miR-22-3p, | miR-133a | | |
| | miR-16-5p, | | | |
| | miR-15b-3p, | | | |
| | miR-29c-3p, | | | |
| | miR-140-3p, | | | |
| | miR-29b-3p, | | | |
| | miR-210, | | | |
| | miR-24-3p, | | | |
| | miR-222-3p, | | | |
| | miR-425-5p, | | | |
| | miR-324-5p | | | |
| Montani et al, miR-Test: | miR-92a-3p, miR-30b-5p, | | Serum, 48 | Starting |
| a blood test for lung | miR-191-5p, miR-484, miR-328-3p, | | NSCLC, 984C | with 34 |
| cancer early detection. | miR-30c-5p, miR-374a-5p, let-7d-5p, | | | miRNA |
| | miR-331-3p, miR-29a-3p, | | | (qPCR) |
| | miR-148a-3p, miR-223-3p, | | | |
| | miR-140-5p | | | |
| Nadal et al, A Novel Serum | miR-141, | | Serum, 154 | Starting with |
| 4-microRNA Signature for | miR-200b, | | NSCLC, 45 C | TaqMan array |
| Lung Cancer Detection | miR-193b, | | | |
| | miR-301 | | | |

In the invention, five specific diagnostic markers are explicitly identified to be suitable for non-small cell lung cancer in Asian and Caucasian population, based on verification by a large number of samples, showing a higher population specificity in relative to other miRNA markers reported internationally. All of these five miRNA diagnostic markers are first proposed and have higher sensitivity and specificity than other miRNA molecular markers.

### BRIEF DESCRIPTION OF THE FIGURES

FIG. 1 is an experimental design flowchart showing the screening, training and verification stages during screening miRNA markers for non-small cell lung cancer according to the present invention.

FIG. 2 is a step diagram for determining the method of the present invention for diagnosing miRNA markers in the serum of patients with non-small cell lung cancer. The control groups comprise healthy or pulmonary inflammatory subjects.

FIG. 3 is a heat map of the expression level of all reliably detected 272 miRNAs. The heat map represents all miRNAs that can be reliably detected; the expression level of miRNAs (copies/ml) is presented on a log 2 scale and normalized to a zero mean. The color of the dot represents the concentration. Hierarchical clustering is performed for two dimensions (miRNAs and samples) based on Euclidean distance. For the horizontal dimension, color is used to represent the patient-control subjects.

FIG. 4 is a heat map of the expression level of 29 differentially expressed miRNAs in the R&D cohort. The expression level of miRNAs (copies/ml) is presented on a log 2 scale and normalized to a zero mean. The color of the dot represents the concentration. Hierarchical clustering is performed for two dimensions (miRNAs and samples) based on Euclidean distance. For the horizontal dimension, color is used to represent the patient-control subjects.

FIG. 5 is a bar chart showing the mean AUCs obtained from the cross validation of various multivariant biomarker panels comprising different numbers of miRNAs selected from hsa-miR-1291, hsa-miR-1-3p, hsa-miR-214-3p, hsa-miR-375 and hsa-let-7a-5p. The error bars represent the standard deviation of the AUC measured.

FIG. 6 is a ROC plot of miRNA marker combinations in each cohort.

FIG. 7 is a box plot of the expression level of miRNA marker combinations in each cohort (control and cancer).

### DETAILED DESCRIPTION OF THE INVENTION

The technical solutions of the present invention will be further specifically described below through specific examples.

In the present invention, unless otherwise specified, the used materials, equipment, and the like are all commercially available or commonly used in the art. The methods in the following examples, unless otherwise stated, are all conventional ones in the art.

The applicants have discovered miRNA markers in study that can be used for diagnosis of non-small cell lung cancer, by which non-small cell lung cancer can be reliably identified.

All miRNA sequences disclosed in the present invention have been stored in the miRBase database (http://www.mirbase.org/).

TABLE 1

| miRNA | Accession | Sequence (5′ → 3′) |
|---|---|---|
| hsa-miR- | MIMAT0005881 | uggcccugacugaagac |
| 1291 | | cagcagu (SEQ ID No. 1) |
| hsa-miR- | MIMAT0000416 | uggaauguaaagaagua |
| 1-3p | | uguau (SEQ ID No. 2) |
| hsa-miR- | MIMAT0000271 | acagcaggcacagacag |
| 214-3p | | gcagu (SEQ ID No. 3) |
| hsa-miR- | MIMAT0000728 | uuuguucguucggcucg |
| 375 | | cguga (SEQ ID No. 4) |
| hsa-let- | MIMAT0000062 | ugagguaguagguugua |
| 7a-5p | | uaguu (SEQ ID No. 5) |

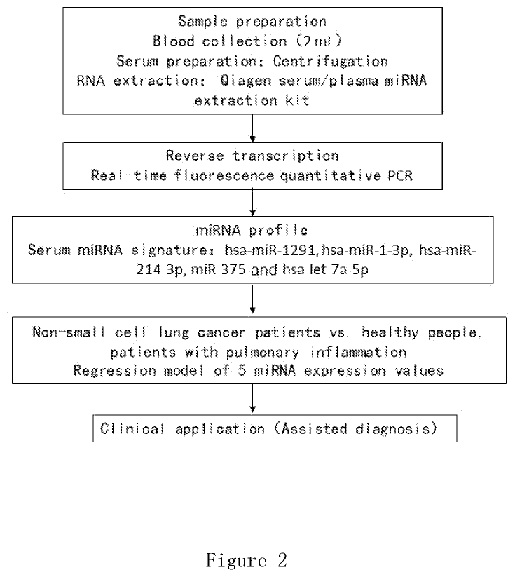

The invention discloses a method for determining diagnostic markers for non-small cell lung cancer (FIG. 2), comprising:
a. performing high-throughput measurement of the expression level of a plurality of miRNAs in a certain number of serums from patients with non-small cell lung cancer;
b. determining the expression level of a plurality of miRNAs in a certain number of control serums; and
c. comparing the relative expression level of the multiple miRNAs in a and b, screening one or more differentially expressed miRNAs as diagnostic markers for non-small cell lung cancer to identify testers' serum.

### EXAMPLES

I. Serum Sample Requirements, Collection and Preparation in R&D Cohort

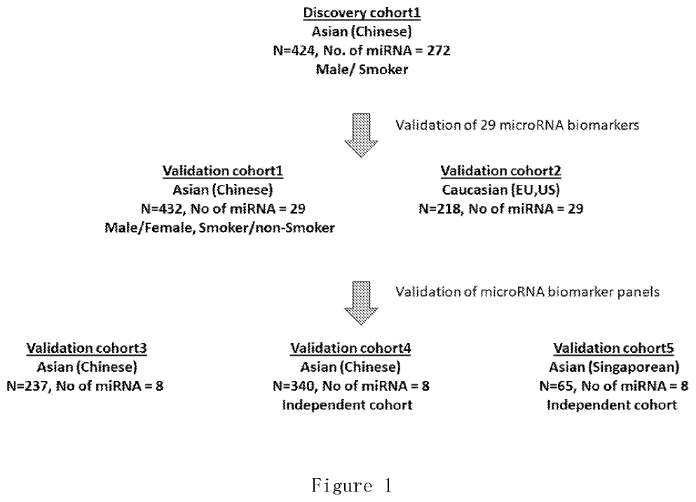

Six cancer case-control cohorts were used in this study to discover and validate biomarkers and biomarker combinations thereof for the detection of early stage lung cancer (FIG. 1). Lung cancer cases in the R&D cohort were from Zhejiang Cancer Hospital of China, and control samples were from the LDCT urban screening project for lung cancer in Keqiao, Zhejiang, China. Subjects who smoked more than 10 packs per year were defined as smokers. In order to match the age of the study group and control subjects as much as possible, only subjects aged between 40 and 85 years were included in the study.

In the experimental design, 200 μL, serum was extracted and total RNA was reverse transcribed, and the amount of cDNA was increased by pre-amplification, whereas the relative expression level of miRNAs remained unchanged. The pre-amplified cDNA was diluted for qPCR measurement. If the miRNA expression concentration was less than 500 copies/ml, it was excluded from the analysis and was considered to be undetectable in subsequent studies.

II. Reverse Transcription-Real-Time Fluorescent PCR Procedure and Results

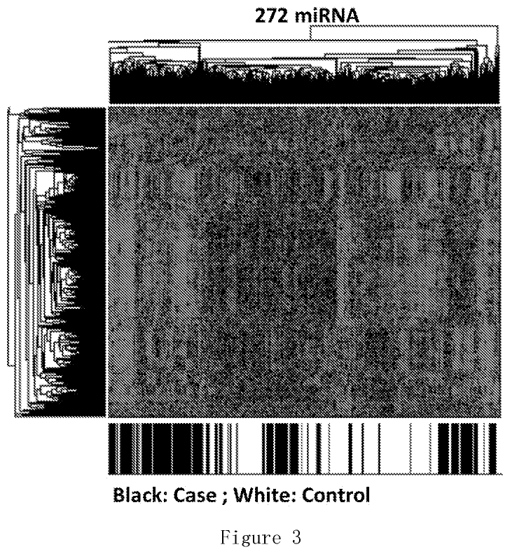

The present invention used RT-qPCR technology to detect the specific expression of 520 candidate miRNAs in serum samples. A standard curve of artificially synthesized miRNA was used to determine copies per ml of serum sample. Among them, 272 miRNAs were reliably detected in more than 90% of samples (with an expression level≥500 copies/ml) (FIG. 3). This was a higher number of miRNAs than previously reported studies using other techniques, highlighting the importance of using an excellent experimental design and well-controlled workflow. The receiver operating characteristic curve (ROC) was used to represent the characteristics of an individual miRNA or a panel of multiple individual biomarkers. The sequential forward floating search (SFFS) algorithm was used to optimize the selection for miRNA biomarkers, and the area under the curve (AUC) value was used to select the optimal marker. A logistic regression equation was used to construct a multi-degree-of-freedom biomarker panel to distinguish between control and cancer groups.

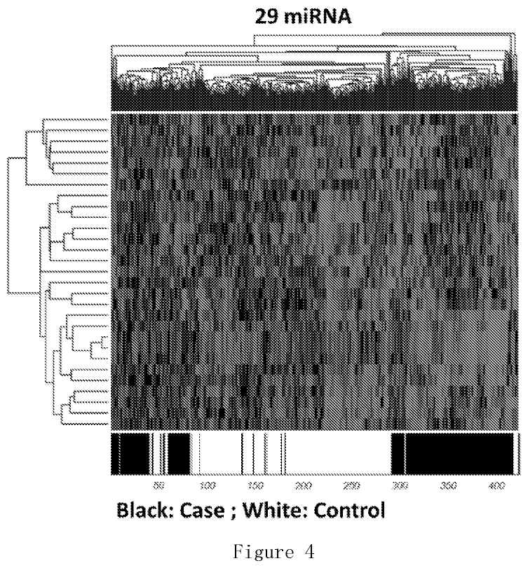

Further studies revealed an individual miRNA biomarker for NSCLC detection. After correction, 29 miRNAs were found to have a p-value less than 0.01, and the difference between the cancer group and control exceeded 1 absolute standard score, with 22 up-regulated and 7 down-regulated in NSCLC subjects. These 29 miRNAs were extracted in the R&D cohort for hierarchical clustering, and a clear grading between cancer and control subjects was observed (FIG. 4). No significant differences were observed among various stages of the non-small cell lung cancer cases. Therefore, in the validation cohorts, these 29 candidate miRNA biomarkers would continue to be validated.

III. Verification of the above 29 miRNAs in Validation Cohorts

The present invention continues to detect these 29 serum miRNA biomarkers using two matched patient-control cohorts. In validation cohort 1,423 cancer and control samples are from the same source as the R&D cohort, but the target population was expanded to males, females, smokers, and non-smokers. In validation cohort 2, the sample included 218 Eastern European males, females, smokers, and non-smokers. The above two validation cohorts included only early-stage (stages 1 and 2) non-small cell lung cancer samples. MiRNA markers less than 0.4 were not significant. 3 up-regulated miRNAs with a p-value less than 0.01 and an absolute standard score greater than 0.4 in both validation cohorts were further selected as biomarkers for non-small cell lung cancer detection (hsa-miR-1291, hsa-miR-1-3p, hsa-miR-214-3p).

IV. Verification of the Above Candidate miRNAs in Validation Cohorts

The present invention further used three additional validation cohorts to validate these three miRNA biomarkers (hsa-miR-1291, hsa-miR-1-3p, hsa-miR-214-3p). Validation cohort 3 comprised of 237 Chinese cancer and control samples which were from the same source as the R&D cohort and validation cohort 1. Validation cohort 4 comprised of 340 independent cancer and control samples. Validation cohort 5 comprised of 65 Singaporean samples. In order to predict non-small cell lung cancer more accurately, the use of biomarkers combinations may be advantageous.

| | | Discovery | | | | | | | | | | | |
|---|---|---|---|---|---|---|---|---|---|---|---|---|---|
| | | (R&D) | | Validation | | Validation | | Validation | | Validation | | Validation | |
| | | cohort 1 | | cohort 1 | | cohort 2 | | cohort 3 | | cohort 4 | | cohort 5 | |
| Ethnicity | | Asian | | Asian | | Caucasian | | Asian | | Asian | | Asian | |
| | | (Chinese) | | (Chinese) | | (European | | (Chinese) | | (Chinese) | | (Singaporean) | |
| | | | | | | and | | | | | | | |
| | | | | | | American) | | | | | | | |
| Grouping | | Con- | Pa- | Con- | Pa- | Con- | Pa- | Con- | Pa- | Con- | Pa- | Con- | Pa- |
| | | trol | tients | trol | tients | trol | tients | trol | tients | trol | tients | trol | tients |
| No. of | | 220 | 204 | 190 | 242 | 117 | 101 | 117 | 120 | 273 | 67 | 31 | 34 |
| subjects | | | | | | | | | | | | | |
| Median age | | 60.55 | 56.9 | 64.0 | 63.0 | 55.0 | 60.0 | 67.0 | 67.0 | 51 | 61 | 60.5 | 63.5 |
| Age range | | 48-70 | 41-75 | 44-77 | 44-77 | 45-64 | 45-75 | 46-77 | 46-77 | 40-68 | 35-76 | 40-71 | 46-85 |
| Gender | Male | 220 | 204 | 150 | 200 | 88 | 80 | 117 | 120 | 121 | 56 | 21 | 25 |
| | Female | 0 | 0 | 40 | 42 | 29 | 21 | 0 | 0 | 152 | 11 | 10 | 9 |
| Smoking | Smoker | 220 | 204 | 150 | 153 | 14 | 152 | 117 | 120 | 273 | 61 | — | — |
| status | Non- | 0 | 0 | 40 | 89 | 109 | 17 | 0 | 0 | 0 | 19 | — | — |
| | smoker | | | | | | | | | | | | |
| Stage | S1 | — | 104 | — | 138 | — | 71 | — | 65 | — | S1 | — | S1 |
| | S2 | — | 76 | — | 104 | — | 30 | — | 55 | — | and | — | and |
| | | | | | | | | | | | S2 | | S2 |
| | S3 | — | 24 | — | 0 | — | 0 | — | 0 | — | | — | |

Hsa-miR-375 and hsa-let-7a-5p are miRNAs for which the expression level were also shown to be significantly down-regulated between the cancer and control samples. The inclusion of these biomarkers to a multivariate panel that may include the novel biomarkers hsa-miR-1291, hsa-miR-1-3p and hsa-miR-214-3p were found to significantly improve the AUC values in at least some of the multivariate panels assessed. The following table provides the AUC, sensitivity, specificity, positive predictive value (PPV) and negative predictive value (NPV) for the individual miRNAs;

| | AUC | Sensitivity | Specificity | PPV | NPV |
|---|---|---|---|---|---|
| hsa-miR-1291 | 0.808 | 0.737 | 0.754 | 0.747 | 0.744 |
| hsa-miR-1-3p | 0.818 | 0.606 | 0.932 | 0.897 | 0.706 |
| hsa-miR-214-3p | 0.810 | 0.650 | 0.859 | 0.820 | 0.714 |
| hsa-miR-375 | 0.751 | 0.555 | 0.859 | 0.795 | 0.663 |
| hsa-let-7a-5p | 0.800 | 0.724 | 0.746 | 0.737 | 0.733 |

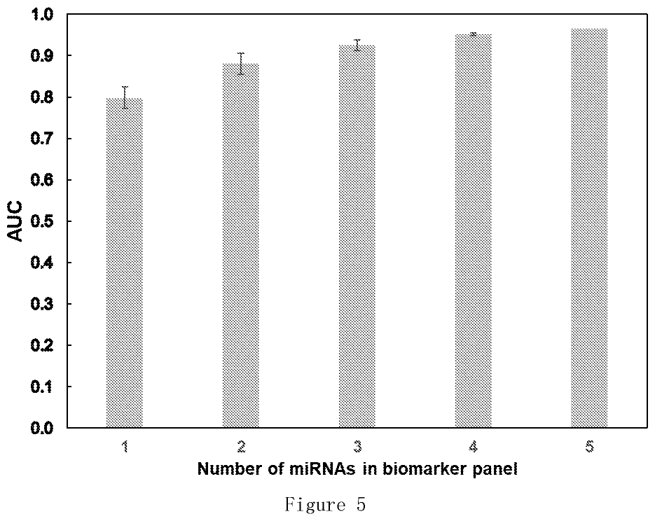

A combination of hsa-miR-1291, hsa-miR-1-3p, hsa-miR-214-3p, hsa-miR-375 and hsa-let-7a-5p was assessed next. FIG. 5 provides the tabulated results of the average AUC values obtained from the analysis of samples in the discovery and validation phases using either the miRNAs individually or as part of 2-, 3-, 4- or 5-miRNA panels. The table below further provides the mean values of the AUC, sensitivity, specificity, positive predictive value (PPV) and negative predictive value (NPV) for the single-miRNAs or for various multivariant biomarker panels analyzed during the cross-validation process. For the 5-miRNA panel, the values provided in the table below represent the actual AUC, sensitivity, PPV and NPV values rather than a mean value (there being only a single possible combination of the five miRNA). It can be concluded that the use of individual miRNAs already demonstrated good diagnostic performance and the diagnostic value of these biomarkers were further enhanced when combined in multivariate panels of up to five miRNAs.

| | Mean | Mean | Mean | Mean | Mean |
|---|---|---|---|---|---|
| | AUC | sensitivity | specificity | PPV | NPV |
| 1-miRNA | 0.798 | 0.654 | 0.830 | 0.799 | 0.712 |
| 2-miRNAs | 0.881 | 0.748 | 0.889 | 0.871 | 0.784 |
| 3-miRNAs | 0.926 | 0.822 | 0.904 | 0.896 | 0.839 |
| 4-miRNAs | 0.951 | 0.848 | 0.936 | 0.931 | 0.863 |
| 5-miRNAs | 0.965 | 0.887 | 0.937 | 0.933 | 0.893 |

The table below further provided the average AUC values of multivariate panels comprising 2-, 3- or 4-miRNAs wherein one miRNA is selected from either hsa-miR-1291, hsa-miR-1-3p, hsa-miR-214-3p, hsa-miR-375 and hsa-let-7a-5p. It is apparent that any of the five miRNAs could be the basis of a multivariate panel with good diagnostic performance.

| | Mean AUC | | |
|---|---|---|---|
| | 2-miRNA | 3-miRNA | 4-miRNA |
| | panel | panel | panel |
| hsa-miR-1291 | 0.879 | 0.923 | 0.950 |
| hsa-miR-1-3p | 0.885 | 0.927 | 0.951 |
| hsa-miR-214-3p | 0.885 | 0.927 | 0.951 |
| hsa-miR-375 | 0.868 | 0.923 | 0.951 |
| hsa-let-7a-5p | 0.885 | 0.929 | 0.953 |

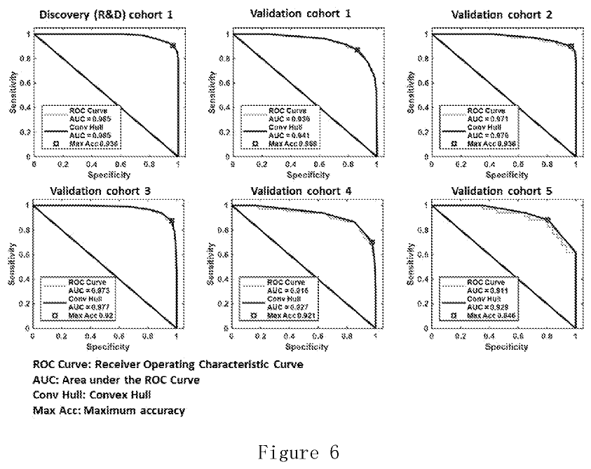

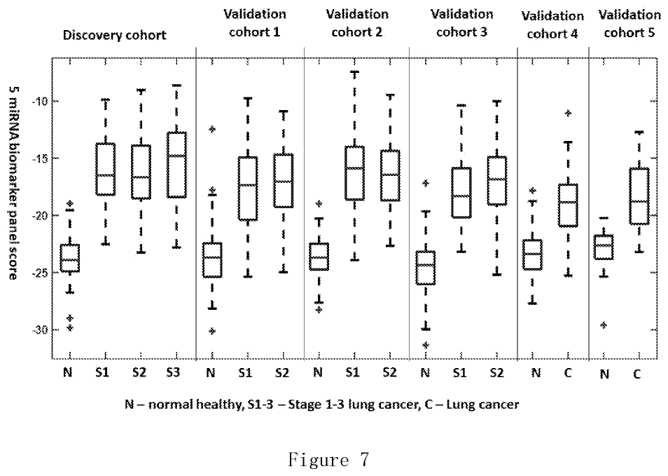

The diagnostic efficacy of the 5-miRNA marker combination (hsa-miR-1291, hsa-miR-1-3p, hsa-miR-214-3p, hsa-miR-375, hsa-let-7a-5p) in the R&D and validation cohorts is shown in FIGS. 6 and 7. Overall, the combination of these five miRNA markers is used to detect non-small cell lung cancer with 80% sensitivity and 90% specificity. FIG. 7 shows the score of samples in each cohort calculated using a combination of five miRNA markers. A good discrimination can be made between non-small cell lung cancer and healthy control populations.

The present invention establishes a complete workflow for discovering and validating serum miRNA biomarker combinations, and has successfully identified biomarkers and a combination thereof for detecting non-small cell lung cancer.

In certain aspects, a patient diagnosed as having lung cancer may receive treatment determined to be appropriate by a medical practitioner. The treatment may include surgery to remove some or all of the malignancy (for example, by pneumonectomy, lobectomy or segmentectomy); ablation of the tumor via radiofrequency ablation (RFA) or radiation therapy; chemotherapy (for example, by administering a therapeutically effective amount of cisplatin, carboplatin, docetaxel, paclitaxel, gemcitabine, vinorelbine, irinotecan, etoposide, vinblastine, pemetrexed, or any combination thereof); targeted therapy (for example, an antibody-based therapy, such as administration of bevacizumab and/or ramucirumab); immunotherapy (for example, by administration of one or more immune checkpoint inhibitors, such as nivolumab, Ipilimumab, pembrolizumab, atezolizumab or durvalumab); and any combination thereof. Palliative treatments may also be used to treat symptoms of the lung cancer. Treatment of lung cancer patients at early stages of the disease showed significant survival benefits. Surgery is the treatment of choice for patients with early stage lung cancer and these patients often demonstrated good survival rates, with a 5-year survival rate of about 75% for patients with Stage 1a lung cancer (Lazdunski, 2013). Adjuvant chemotherapy or targeted therapy provided for early-stage lung cancer also could be beneficial (Gadgeel, 2017).

The above-mentioned examples are only preferred embodiments of the present invention, and are not intended to limit the present invention in any way, and other variations and modifications are possible without departing from the technical solutions described in the claims.

1. A peripheral blood miRNA marker for diagnosis of non-small cell lung cancer, comprising at least one of hsa-miR-1291, hsa-miR-1-3p, and hsa-miR-214-3p.

2. The peripheral blood miRNA marker for diagnosis of non-small cell lung cancer according to claim 1, further comprising one or both of hsa-miR-375 and hsa-let-7a-5p.

3. The peripheral blood miRNA marker for diagnosis of non-small cell lung cancer according to claim 1, wherein the peripheral blood is serum or plasma.

4. The peripheral blood miRNA marker for diagnosis of non-small cell lung cancer according to claim 1, wherein the expression of the peripheral blood miRNA marker is differentially regulated in the peripheral blood of a patient diagnosed with non-small cell lung cancer compared to that in a control sample.

5. The peripheral blood miRNA marker for diagnosis of non-small cell lung cancer according to claim 4, wherein the control sample is a subject not suffering from non-small cell lung cancer.

6. The peripheral blood miRNA marker for diagnosis of non-small cell lung cancer according to claim 1, wherein the non-small cell lung cancer comprises squamous cell lung cancer, and adenocarcinoma lung cancer.

7. A kit for diagnosis of non-small cell lung cancer, characterized in that the kit comprises at least one reagent for detecting a peripheral blood miRNA according to claim 1.

8. Use of the peripheral blood miRNA marker according to claim 1, in the preparation of a diagnostic agent for non-small cell lung cancer for predicting the possibility for a subject to develop or have non-small cell lung cancer by a method, the method comprising:
detecting the presence of miRNAs in a peripheral blood sample obtained from the subject;
measuring the expression level of at least one miRNA selected from the group comprising of hsa-miR-1291, hsa-miR-1-3p and hsa-miR-214-3p, and optionally the expression level of at least one miRNA selected from hsa-miR-375 and hsa-let-7a-5p, in the peripheral blood sample; and
using a score based on the previously measured miRNA expression level to predict the possibility that the subject will develop or have non-small cell lung cancer.

9. Use according to claim 8, wherein the score of the miRNA expression level is calculated using a classification algorithm selected from the group consisting of: support vector machine algorithm, logistic regression algorithm, multinomial logistic regression algorithm, Fisher's linear discriminant algorithm, quadratic classifier algorithm, perceptron algorithm, k-nearest neighbor algorithm, artificial neural network algorithm, random forest algorithm, decision tree algorithm, naive Bayes algorithm, adaptive Bayes network algorithm, and an integrated learning method that combines multiple learning algorithms.

10. Use according to claim 9, wherein the classification algorithm is pre-trained using the expression level of a control.

11. Use according to claim 10, wherein the control is at least one selected from the group consisting of a control without non-small cell lung cancer and a non-small cell lung cancer patient.

12. Use according to claim 10, wherein the classification algorithm compares the expression level in the subject with that in the control and returns a mathematical score that identifies the possibility that the subject belongs to any one of the control groups.

13. Use according to claim 8, wherein the expression level of the miRNAs is in any one of concentration, log(concentration), Ct/Cq, and Ct/Cq power of 2.

14. Use according to claim 8, wherein the non-small cell lung cancer comprises non-small cell lung cancer in various stages.

15. Use according to claim 8, wherein the subject comprises, but is not limited to, Asians and Caucasians.

16. The peripheral blood miRNA marker for diagnosis of non-small cell lung cancer according to claim 2, wherein the expression of the peripheral blood miRNA marker is differentially regulated in the peripheral blood of a patient diagnosed with non-small cell lung cancer compared to that in a control sample.

17. The peripheral blood miRNA marker for diagnosis of non-small cell lung cancer according to claim 16, wherein the control sample is a subject not suffering from non-small cell lung cancer.

18. The peripheral blood miRNA marker for diagnosis of non-small cell lung cancer according to claim 2, wherein the non-small cell lung cancer comprises squamous cell lung cancer, and adenocarcinoma lung cancer.

19. The kit according to claim 7, wherein the kit further comprises at least one reagent for detecting a peripheral blood miRNA selected from hsa-miR-375 and hsa-let-7a-5p.
